(19) United States
(12) Patent Application Publication
(10) Pub. No.: US 2025/0145937 A1
(43) Pub. Date: May 8, 2025

(54) MICROALGA DISPERSION AND PREPARATION METHOD OF MICROALGA DISPERSION

(71) Applicant: DAICEN MEMBRANE-SYSTEMS Ltd., Tokyo (JP)

(72) Inventors: Kenichi NISHIZAKA, Tokyo (JP); Shuji NAKATSUKA, Tokyo (JP); Yoji INOUE, Tokyo (JP); Shuusuke YOKOTA, Tokyo (JP)

(73) Assignee: DAICEN MEMBRANE-SYSTEMS Ltd., Tokyo (JP)

(21) Appl. No.: 18/835,865

(22) PCT Filed: Feb. 22, 2023
(86) PCT No.: PCT/JP2023/006447

§ 371 Date: Aug. 5, 2024

(30) Foreign Application Priority Data
Mar. 9, 2022 (JP) 2022-036202

(51) Int. Cl. C12N 1/12; C12N 1/04

(52) U.S. Cl. CPC C12N 1/12; C12N 1/04

(57) ABSTRACT

A method for preparing a microalgal dispersion from a microalga-containing liquid, the microalga-containing liquid containing a microalga having a body length of less than 1000 μm, the method including a concentration step of performing cross flow filtration of the microalga-containing liquid using a hollow fiber membrane, wherein a circulation linear velocity in the cross flow filtration in the concentration step is in a range of 0.2 m/sec or more and 1.3 m/sec or less.

### TECHNICAL FIELD

The present disclosure relates to a microalgal dispersion having an increased cell concentration of microalgae, which can be used in biofuels, pharmaceuticals, supplements, cosmetics, chemical products, foods, feeds, fertilizers, and the like, and a method for preparing a microalgal dispersion.

### BACKGROUND ART

For carbon neutrality, attempts have been made to culture microalgae (phytoplankton) that perform photosynthesis in large quantities and to use them as biofuels and the like.

JP 2016-49514 A describes an invention of a method for recovering a suspended substance, the method including a concentration step of concentrating a liquid to be treated containing a suspended substance.

The document JP 2016-49514 A describes that the concentration step includes a filtration step, a peeling step, and a drawing step; that the drawing step is performed either sequentially after the filtration step and the peeling step are sequentially performed; or in parallel with the filtration step or the peeling step or separately from the filtration step and the peeling step while the filtration step and the peeling step are sequentially repeated. The document JP 2016-49514 A also describes that cross flow washing of a filter medium is not performed in the concentration step, and that the suspended substance is a microorganism (claims).

JP H03-39084 A describes a method for concentrating a single-cell alga culture solution. The method includes performing regular underflow washing in cross flow filtration using a hollow-fiber ultrafiltration membrane (UF membrane) module having a molecular weight cutoff of from 10000 to 1000000 to concentrate the single-cell alga culture solution (claims).

### SUMMARY OF INVENTION

An object of the present disclosure is to provide a microalgal dispersion having an increased cell concentration of a microalga, a frozen product thereof, a valuable product using the microalgal dispersion or the frozen product as a raw material, a method for preparing a microalgal dispersion, and a method for producing a valuable product using a microalgal dispersion obtained by the method for preparing a microalgal dispersion or a frozen product as a raw material.

An embodiment of the present disclosure provides a microalgal dispersion containing a microalga having a body length of less than 10 μm, the microalgal dispersion having a cell concentration of 2 billion cells/cc or more, and a valuable product containing a frozen product of the microalgal dispersion.

The present disclosure also provides a valuable product containing a microalgal dispersion containing a microalga having a body length of less than 1000 μm, the microalgal dispersion having a cell concentration of 2 billion cells/cc or more; a useful component produced by the microalga contained in the dispersion; a frozen product of the microalgal dispersion; or a useful component produced by the microalga contained in the frozen product, wherein the valuable product is selected from a biofuel, a pharmaceutical, a supplement, a cosmetic, a chemical product, a food, a feed, and a fertilizer.

The present disclosure also provides a method for preparing a microalgal dispersion from a microalga-containing liquid, the microalga-containing liquid containing a microalga having a body length of less than 1000 μm, the method including a concentration step of performing cross flow filtration of the microalga-containing liquid using a hollow fiber membrane, wherein a circulation linear velocity in the cross flow filtration in the concentration step is in a range of 0.2 m/sec or more and 1.3 m/sec or less. Concentration is performed, for example, until a cell concentration of the microalga reaches 2 billion cells/cc or more.

The present disclosure also provides a method for producing a valuable product, the method including producing a microalgal dispersion by the method for preparing a microalgal dispersion; charging the microalgal dispersion as a production raw material; and kneading and mixing the production raw material, wherein the valuable product is selected from a biofuel, a pharmaceutical, a supplement, a cosmetic, a chemical product, a food, a feed, and a fertilizer.

The microalgal dispersion according to an embodiment of the present disclosure has an increased cell concentration, and thus can be applied to various applications such as a biofuel, a pharmaceutical, a supplement, a cosmetic, a chemical product, a food, a feed, and a fertilizer.

### BRIEF DESCRIPTION OF DRAWINGS

FIG. 1 is a production flow diagram for explaining a method for preparing a microalgal dispersion according to an embodiment of the present disclosure.

FIG. 2 is a diagram illustrating measurement results of Referential Example 1 and Referential Example 2.

FIG. 3 is a diagram illustrating measurement results of Referential Example 1 and Referential Example 2.

### DESCRIPTION OF EMBODIMENTS

### Microalgal Dispersion and Frozen Product Thereof

The microalgal dispersion according to an embodiment of the present disclosure is a dispersion containing a microalga having a body length of less than 10 μm. The solvent may be water such as fresh water, brackish water, seawater, or deep sea water that is close to the environment in which the microalga of interest inhabits.

A microalga (phytoplankton) is a small living organism having a diameter of 1000 μm or less and a single cell (one cell) as a unit, and proliferates by division. A microalga has chlorophyll and is capable of producing oxygen by fixing carbon dioxide in the atmosphere by photosynthesis as in general plants.

Examples of the microalga contained in the microalgal dispersion according to an embodiment of the present disclosure include microalgae having a body length of less than 10 μm, such as chlorella, dunaliella, nannochloropsis, Pseudochoricystis ellipsoidea, and Cyanidioschyzon merolae. Here, the body length of a microalga is the longest diameter of a circle, an oval, an ellipse, or an egg-like shape when one individual is projected, and, in the case of a microalga having a flagellum or pilus, the length of the flagellum or pilus is not included in the body length.

In the microalgal dispersion according to an embodiment of the present disclosure, when the body length of the microalga is less than 10 μm, the cell concentration thereof is 2 billion cells/cc or more. The cell concentration is preferably 3 billion cells/cc or more, more preferably 5 billion cells/cc or more, even more preferably 8 billion cells/cc or more, and still more preferably 10 billion cells/cc or more. The microalgal dispersion according to an embodiment of the present disclosure may be a dispersion of a microalga having a body length of from 2 to 5 μm, and may have a cell concentration of 10 billion cells/cc or more.

The microalga is preferably nannochloropsis, chlorella, or the like having a body length of less than about 5 μm. When the microalgal dispersion according to an embodiment of the present disclosure contains nannochloropsis or chlorella, the cell concentration is preferably 11 billion cells/cc or more, more preferably 12 billion cells/cc or more, even more preferably 14 billion cells/cc or more, and still more preferably 15 billion cells/cc.

The cell concentration has a limit value depending on the body length of the microalga, and when the cell concentration is excessively increased, the risk of quality deterioration such as rot due to breakage of a cell membrane increases. According to an embodiment of the present disclosure, the cell concentration can be appropriately selected depending on the purpose of a concentration operation.

The method for measuring the cell concentration is not particularly limited, and the cell concentration can be measured by a known measurement method such as a fluorescence method or a turbidity/absorbance method. In particular, it is preferable to statistically measure the cell concentration by visually counting the number of cells in a precise diluted liquid with a microscope, using a hemocytometer, which is a laboratory instrument capable of counting the number of cells such as red blood cells, white blood cells, sperm cells, or cultured cells.

When the cell concentration is less than 30 million cells/cc, the cell concentration can be measured, without dilution, using a hemocytometer. The hemocytometer includes a slide glass having a scale in units of micrometers, and the number of cells in a counting chamber, which is a gap between the slide glass and a cover glass, can be counted under a microscope. There are several types of hemocytometers, such as a Thoma hemocytometer, a Bürker-Türk hemocytometer, an improved Neubauer hemocytometer, and a Fuchs-Rosenthal hemocytometer, depending on how the scale is engraved and the depth of the counting chamber.

The dispersion containing a microalga according to an embodiment of the present disclosure has a solid content concentration of 5% or more. The solid content concentration of the microalgal dispersion according to an embodiment of the present disclosure is a non-volatile component concentration in an absolutely dry state in which moisture, including moisture of the microalga itself, is volatilized. The solid content concentration can be appropriately designed in accordance with the purpose of the present disclosure, and is, for example, preferably 5.0% or more, more preferably 6.0% or more, even more preferably 7.0% or more, and most preferably 8.0% or more.

### Measurement of Solid Content Concentration of Microalgal Dispersion

The solid content concentration of the microalgal dispersion and the microalga-containing liquid according to an embodiment of the present disclosure can be measured by a known method. For example, measurement using a generally used constant temperature dryer may be performed.

In an example of measurement using a constant temperature dryer, a liquid to be measured is taken into, for example, an aluminum dish for measurement of moisture subjected to precise weighing in advance, and the mass of a measurement sample is precisely weighed to, for example, four decimal places. In a case where the solid content concentration of the liquid to be measured is expected to be low, the mass of the measurement sample is increased and thus the solid content concentration can be accurately measured.

When the viscosity of the liquid to be measured is high, an appropriate amount of pure water is added and the liquid is sufficiently diluted. The appropriate amount of pure water is added for uniformly spreading the measurement sample on the aluminum dish for measurement of moisture and accurately measuring the solid content concentration. However, since the entire amount of pure water is evaporated and dried in the constant temperature dryer, the pure water does not affect the solid content concentration measurement value. The aluminum dish is placed in the constant temperature dryer and dried at a constant temperature for a constant time, and moisture including intracellular water of the microalga is completely evaporated and dried.

After being removed from the constant temperature dryer, the dried product is cooled to room temperature while moisture absorption is avoided, the mass of the dried product is precisely weighed, and the solid content concentration is calculated, according to the following equation, from a mass after drying obtained by subtracting a precisely weighed mass of the aluminum dish for measurement of moisture.

$$Solid content concentration ( \% ) = ( mass after drying / sample mass ) \times 10$$

Examples of the microalgal dispersion according to an embodiment of the present disclosure include those containing chlorella having a relatively low lipid content (lipid content: 8.9 mass %), or nannochloropsis, dunaliella, or the like having a relatively high lipid content. Nannochloropsis is a group belonging to the class Eustigmatophyceae, the order Eustigmatales, the family Eustigmataceae and the genus Nannochloropsis, and is a spherical unicellular eubacterium having a body length of from 2 to 5 μm and a high EPA (eicosapentaenoic acid) content. Among lipids of nannochloropsis, the EPA content is about 35 mass %, which is higher than the content of palmitic acid (about 20 mass %) or the content of palmitoleic acid (about 27 mass %). Examples of microalgae having a high content of EPA among lipids include phaeodactylum (about 32 mass %) and borphyridium (about 33 mass %). Examples of microalgae having a high DHA (docosahexaenoic acid) content include lapyrinthula (about 43 mass %).

When the microalgal dispersion according to an embodiment of the present disclosure is used as a biofuel, a microalga having a property of producing and accumulating oils and fats upon photosynthesis are suitable. Preferred examples of such microalgae include microalgae classified into the class Chlorophyceae, the class Prasinophyceae, the class Cryptophyceae, and the class Cyanophyceae.

Examples thereof include algae belonging to the genus Pseudochoricystis (Pseudochoricystis) including Pseudochoricystis ellipsoidea (Pseudochoricystis ellipsoidea); algae belonging to the genus Choricystis (Choricystis); algae belonging to the genus Chlamydomonas (Chlamydomonas) including Chlamydomonas reinhardtii (Chlamydomonas reinhardtii); algae belonging to the genus Cyanidioschyzon (Cyanidioschyzon) including Cyanidioschyzon merolae (Cyanidoschyzon merolae), or algae belonging to the genus Cyanidium (Cyanidium) including Cyanidium caldarium (Cyanidium caldarium).

Pseudochoricystis ellipsoidea is a freshwater green alga having an oval or slightly curved kidney shape and a cell length of from 3 to 4 μm. The alga is characterized in that it proliferates rapidly while it is rich in inorganic nutrients (nitric acid, phosphoric acid, potassium, etc.), whereas, when the inorganic nutrients are depleted, it absorbs water and carbon dioxide through photosynthesis and accumulates large amounts of oils and fats in its cells. The accumulated oils and fats are saturated or unsaturated aliphatic hydrocarbons having from 10 to 25 carbons and correspond to light oils.

The genus Chlamydomonas includes unicellular flagellates belonging to the class Chlorophyceae. Among them, Chlamydomonas reinhardtii belonging to the genus Chlamydomonas is widely known as a model organism in the field of molecular biology and the like. The appearance of the cells is a smooth ellipse with a length of about from 10 to 20 μm.

Cyanidioschyzon merolae and Cyanidium caldarium are algae belonging to the class Rhodophyceae, and are known as hot spring algae living in the vicinity of hot spring sources or in bathtubs. Cyanidioschyzon merolae cells have a diameter of about from 1 to 2 μm. In addition, Cyanidium caldarium cells are spherical and have a diameter of from several micrometers to several tens of micrometers.

The frozen product of the microalgal dispersion according to an embodiment of the present disclosure is produced by freezing and solidifying the above-described microalgal dispersion by a known freezing method.

Examples of the known freezing method include a method of freezing at a low temperature by a known freezer, and a method of freezing by a freezer and then freeze-drying.

Since the microalgal dispersion or the frozen product thereof according to an embodiment of the present disclosure has an increased cell concentration, transportation costs can be reduced. In a case where the microalgal dispersion or the frozen product thereof is used as a raw material for products such as biofuels, pharmaceuticals, supplements, cosmetics, chemical products, foods, feeds, and fertilizers, the influence of a solvent such as water on the products when the solvent is charged in a production process can be suppressed, which is preferred.

The microalgal dispersion or the frozen product thereof (including a useful component produced by the microalga) according to an embodiment of the present disclosure is suitable as a production raw material for products containing a large amount of lipids, particularly unsaturated fatty acids such as DHA (docosahexaenoic acid) and EPA (eicosapentaenoic acid), in applications of biofuels, pharmaceuticals, supplements, cosmetics, chemical products, foods, feeds, and fertilizers.

The microalgal dispersion or the frozen product thereof according to an embodiment of the present disclosure is preferable as a production raw material particularly for an application in which the amount of a lipid component is important among the above-described applications. The lipid content of the microalga is preferably 15 mass % or more, and more preferably 20 mass % or more.

### Valuable Product

The valuable product according to an embodiment of the present disclosure is selected from biofuels, pharmaceuticals, supplements, cosmetics, chemical products, foods, feeds, and fertilizers.

The valuable product according to an embodiment of the present disclosure may contain the microalgal dispersion or the frozen product thereof, or may contain a useful component produced by the microalga.

The valuable product according to an embodiment of the present disclosure can contain a dispersion containing a microalga having a body length of less than 1000 μm, the dispersion having a solid content concentration of 5% or more, a useful component produced by the microalga contained therein, a frozen product of the microalgal dispersion, or a useful component produced by the microalgae contained therein.

The microalga in the microalgal dispersion or the frozen product thereof contained in the valuable product according to an embodiment of the present disclosure has a body length of less than 1000 μm. Here, the body length of a microalga is the longest diameter of a circle, an oval, an ellipse, or an egg-like shape when one individual is projected, and, in the case of a microalga having a flagellum or pilus, the length of the flagellum or pilus is not included in the body length. The microalga in the microalgal dispersion or the frozen product thereof contained in the valuable product according to an embodiment of the present disclosure preferably has a body length of less than 10 μm.

The microalga in the microalgal dispersion or the frozen product thereof contained in the valuable product according to an embodiment of the present disclosure has a cell concentration of 2 billion cells/cc or more. The cell concentration is preferably 3 billion cells/cc or more, more preferably 5 billion cells/cc or more, even more preferably 8 billion cells/cc or more, and still more preferably 10 billion cells/cc or more. The microalgal dispersion according to an embodiment of the present disclosure may be a dispersion of a microalga having a body length of from 2 to 5 μm, and may have a cell concentration of 10 billion cells/cc or more.

The microalga is preferably a microalga having a body length of less than 500 μm and selected from spirulina, chlorella, euglena, pavlova, haematococcus, dunaliella, nannochloropsis, phaeodactylum, borphyridium, lapyrinthula, algae belonging to the genus Pseudochoricystis including Pseudochoricystis ellipsoidea; algae belonging to the genus Choricystis; algae belonging to the genus Chlamydomonas including Chlamydomonas reinhardtii; algae belonging to the genus Cyanidioschyzon including Cyanidioschyzon merolae, or algae belonging to the genus Cyanidium including Cyanidium caldarium. The microalga is more preferably a microalga having a body length of less than 10 μm and selected from chlorella, dunaliella, nannochloropsis, Pseudochoricystis ellipsoidea, and Cyanidioschyzon merolae.

The invention of JP 2016-49514 A essentially includes a peeling step of depositing a microalga on an MBR membrane surface and scraping off the microalga. When the peeling step is carried out, a cell membrane is damaged by peeling (scraping), and peeled materials are adhered to each other due to adhesiveness of a cell sap, which makes it difficult to handle the peeled materials. Therefore, the peeled materials are dried and not adhered to each other before proceeding to the next step.

In addition, in the peeling step, a filter medium having high abrasion resistance such as PVDF must be selected. The filter medium will cause a new problem that clogging is likely to occur due to fixation and adhesion of the microalga onto the membrane surface. In contrast, the valuable product according to an embodiment of the present disclosure uses the microalgal dispersion or the frozen product thereof, and none of the peeling step, the drawing step, and the drying step are carried out. Therefore, there is an advantage in that the cell membrane is not damaged, that the quality of the valuable product is improved, and that the production process is shortened.

Examples of the useful component contained in the valuable product according to an embodiment of the present disclosure and produced by the microalga can include EPA produced by nannochloropsis.

The valuable product according to an embodiment of the present disclosure may contain an additional component, such as an additive, required depending on the application.

When the valuable product according to an embodiment of the present disclosure is a feed, for example, a frozen product of a nannochloropsis dispersion stored in a large freezer at −20° C. can be directly used as a Brachionus plicatilis feed, which is a biological feed for larval fish, for the purpose of EPA enrichment of farmed fish.

### Method for Preparing Microalgal Dispersion

The microalgal dispersion according to an embodiment of the present disclosure can be prepared by a concentration operation using, for example, a membrane, a centrifuge, or any other physical process such as evaporation, but the preparation method is not limited to such a method.

According to an embodiment of the present disclosure, the microalgal dispersion of the present disclosure may be prepared from a microalga-containing liquid using a hollow fiber membrane. The method for preparing a microalgal dispersion from a microalga-containing liquid according to an embodiment of the present disclosure includes a concentration step of performing cross flow filtration of a microalga-containing liquid containing a microalga having a body length of less than 1000 μm using a hollow fiber membrane. A circulation linear velocity in the cross flow filtration in the concentration step can be in a range of 0.2 m/sec or more and 1.3 m/sec or less.

The hollow fiber membrane used in the concentration step will be described.

As the hollow fiber membrane used in the concentration step, a hollow fiber membrane having a pore diameter that increases a filtration rate of the microalga-containing liquid (liquid to be treated) can be appropriately selected depending on the body length and the like of the microalga contained in the liquid to be treated as long as the microalga does not leak into the filtrate side.

The hollow fiber membrane used in the concentration step can be an ultrafiltration membrane or microfiltration membrane having a pore diameter of about from 0.01 μm to 500 μm.

The material of the hollow fiber membrane used in the concentration step is not particularly limited, and can be an inorganic material or an organic polymer material having an angle of contact with water of less than 60°, for example, cellulose acetate.

Water, when dropped on a solid surface, is rounded by its own surface tension, and an angle θ formed between a tangential line of a water droplet and the solid surface is referred to as angle of contact with water. For example, it is known that the contact angle of cellulose acetate with water is from 50° to 55°, that of polyacrylonitrile is from 55° to 58°, and that of polyether sulfone is from 65° to 70°.

The contact angle of a highly hydrophilic organic polymer material with water is lower than that of a hydrophobic organic polymer material. In an example of the concentration method according to an embodiment of the present disclosure, a decrease in filtration rate during concentration treatment can be reduced by using a hollow fiber membrane made of an organic polymer having an angle of contact with water of less than 60°.

In particular, when a hollow fiber membrane made of cellulose acetate is used, the filtration rate during the concentration treatment decreases less. The reason for this is possibly as follows. The cells of the microalga have a weakly negatively charged surface, and the cellulose acetate membrane also has a weakly negatively charged surface. Thus, the cells and the cellulose acetate membrane are not attracted to each other by a strong Coulombic force. A shear force of the cross flow filtration functions by adjusting the circulation linear velocity in a range of 0.2 m/sec or more and 1.3 m/sec or less. Further, the microalga accumulating on the membrane surface can be peeled off from the membrane surfaces by performing regular underflow washing.

In the measurement of the angle of contact with water, a θ/2 method is generally used, and the radius r and height h of a liquid droplet are determined and substituted into the following formula to determine a contact angle θ.

$$tan θ = h / r \to θ = 2 arctan ( h / r )$$

Furthermore, the contact angle θ can also be measured using a fully automatic contact angle meter (available from Kyowa Interface Science Co., Ltd.).

Examples of the organic polymer having an angle of contact with water of less than 60° include cellulose-based polymers such as cellulose acetate, acrylic acid-based polymers such as polyacrylonitrile, vinyl alcohol-based polymers such as polyethylene vinyl alcohol, polyamide-based polymers, hydrophobic polymers modified with water-soluble polymers such as polyethylene glycol-based polymers such as polyethylene glycol, sodium polyacrylate-based polymers, and polypyrrolidone-based polymers such as polyvinylpyrrolidone, and hydrophilized polymers. The hydrophobic polymer modified with a water-soluble polymer does not require the presence of a covalent bond between a hydrophilic component and a hydrophobic component, and also includes, for example, an organic polymer obtained by a means of mixing and dissolving a hydrophobic polymer and a hydrophilic polymer as film-forming solution components and forming a film as in the case of a hollow fiber membrane described in JP 5065379 B.

According to an embodiment of the present disclosure, in the hollow fiber membrane used in the concentration step, its molecular weight cutoff, which is correlated with the pore diameter of the hollow fiber membrane, may also be appropriately selected. The hollow fiber membrane used in the concentration step is preferably an ultrafiltration membrane having a molecular weight cutoff of from 10000 to 1000000 and more preferably a molecular weight cutoff of from 10000 to 500000, and most preferably a molecular weight cutoff of from 10000 to 300000 in the case of a microalga having a body length of less than 10 μm.

According to an embodiment of the present disclosure, the hollow fiber membrane used in the concentration step has an internal diameter in a range of from 0.5 mm to 2.0 mm. When it is desired to further increase the cell concentration of the microalga in the present disclosure, a hollow fiber membrane having a large internal diameter is advantageous for securing a high circulation linear velocity. However, when the internal diameter is large, the membrane area per module volume decreases, and thus an appropriate internal diameter can be set according to the purpose of the concentration treatment operation.

According to an embodiment of the present disclosure, the cellulose acetate hollow fiber membrane used in the concentration step preferably has an internal diameter of from 0.7 mm to 1.6 mm, and more preferably from 0.8 to 1.4 mm.

According to an embodiment of the present disclosure, the hollow fiber membrane used in the concentration step can be used as a hollow fiber membrane module in which a bundle of a plurality of hollow fiber membranes is accommodated in a case housing.

The microalga-containing liquid serving as the liquid to be treated used in the concentration step will be described.

According to an embodiment of the present disclosure, the microalga-containing liquid used in the concentration step contains a microalga and a solvent. The microalga has a body length of less than 1000 μm. The body length of a microalga is the longest diameter of a circle, an oval, an ellipse, or an egg-like shape when one individual is projected, and, in the case of a microalga having a flagellum or pilus, the length of the flagellum or pilus is not included in the body length.

The microalga is preferably a microalga having a body length of less than 500 μm and selected from spirulina, chlorella, euglena, pavlova, haematococcus, dunaliella, nannochloropsis, phaeodactylum, borphyridium, lapyrinthula, algae belonging to the genus Pseudochoricystis including Pseudochoricystis ellipsoidea; algae belonging to the genus Choricystis; algae belonging to the genus Chlamydomonas including Chlamydomonas reinhardtii; algae belonging to the genus Cyanidioschyzon including Cyanidioschyzon merolae, or algae belonging to the genus Cyanidium including Cyanidium caldarium. The microalga is more preferably a microalga having a body length of less than 10 μm and selected from chlorella, dunaliella, nannochloropsis, Pseudochoricystis ellipsoidea, and Cyanidioschyzon merolae.

The solvent of the microalga-containing liquid is generally water such as fresh water, brackish water, seawater, or deep sea water that is close to the environment in which the microalga of interest inhabits. If necessary, the microalga-containing liquid can also contain an additive such as a dispersion stabilizer other than components of the water in which the target microalga inhabits. Further, the osmotic pressure of the microalga-containing liquid can be appropriately adjusted by a salt concentration.

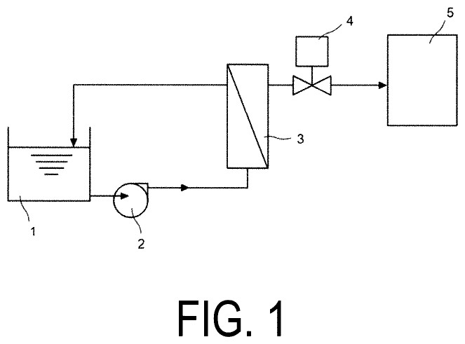

Next, an embodiment of the cross flow filtration in the concentration step will be described with reference to FIG. 1. A stock solution tank 1 contains a microalga-containing liquid (liquid to be treated) serving as a stock solution, for example, a liquid to be treated having a cell concentration of less than 30 million cells/cc.

The liquid to be treated in the stock solution tank 1 is fed to a hollow fiber membrane module 3 by operating a liquid feed pump 2 and is subjected to filtration treatment. In the filtration treatment in the hollow fiber membrane module 3, the cross flow filtration is performed.

Filtered water (permeated water) generated in the hollow fiber membrane module 3 is fed to and stored in a permeated water tank 5. The permeated water tank 5 may be omitted, and the permeated water may be discharged as is or may be used for other purposes such as sprinkling or washing. Reference numeral 4 denotes an automatic on-off valve for performing underflow washing.

The concentrated water generated in the hollow fiber membrane module 3 is returned to the stock solution tank 1. Thus, the cell concentration of the microalga in the liquid to be treated (dispersion) can be increased by repeating the concentration step by the circulation treatment in a circulation line of the stock solution tank 1, the hollow fiber membrane module 3, and the stock solution tank 1.

The circulation linear velocity in the cross flow filtration in the filtration operation (concentration step) of the hollow fiber membrane module 3 will be described. The circulation linear velocity in the internal pressure cross flow filtration corresponds to the volume of circulating water (concentrated water) per second per flow path area inside the hollow fiber membrane, and its unit is m³/m²·sec (m/sec).

In the concentration process, internal pressure cross flow filtration is generally used. However, in the case of external pressure cross flow filtration, the circulation linear velocity is calculated as the volume of circulating water per second per flow path area outside the hollow fiber membrane.

An increase in circulation linear velocity causes an increase in shearing force for peeling off the microalga accumulated on the surface of the hollow fiber membrane from the hollow fiber membrane in the cross flow filtration, which is preferred. Meanwhile, an excessively high value of the circulation linear velocity causes a risk that the cell membrane may be damaged by the shearing force during the concentration treatment, resulting also in an increase in concentration energy amount. Therefore, the circulation linear velocity is preferably in a range of 0.2 m/sec or more and 1.3 m/sec or less.

According to an embodiment of the present disclosure, the circulation linear velocity is more preferably in a range of from 0.3 m/sec to 1.2 m/sec, even more preferably in a range of from 0.4 m/sec to 1.0 m/sec, and still more preferably in a range of from 0.5 m/sec to 0.8 m/sec.

The operating pressure at an inlet during the filtration operation in the hollow fiber membrane module 3 can be adjusted such that the circulation linear velocity can be set to a required value, and the operating pressure can be, for example, within a range of from 0.03 MPa to 0.3 MPa.

The filtration rate in the filtration operation in the hollow fiber membrane module 3 is desirably high, but decreases as the cell concentration of the dispersion increases. The material of the hollow fiber membrane is an organic polymer material having an angle of contact with water of less than 60°, thereby making it possible to suppress a decrease in filtration rate, which is preferred.

In addition, it is possible to suppress a decrease in filtration rate by performing the underflow washing described in JP H03-39084 A. As the underflow washing, it is preferable to perform automatic underflow washing under the condition that the automatic on-off valve is closed for from 20 seconds to 40 seconds at intervals of from 10 minutes to 30 minutes during the filtration operation in the hollow fiber membrane module 3.

The operating pressure at the inlet of the hollow fiber membrane module 3 is preferably in a range of from 0.05 MPa to 0.2 MPa, and an average filtration rate is preferably in a range of from 50 L/m²·Hr to 150 L/m²·Hr.

After the microalgal dispersion having the target cell concentration is obtained by the concentration step, it is preferable to wash the hollow fiber membrane module 3 and return the performance state of the hollow fiber membrane module to the original state to such an extent that a change from the initial state at the start of the concentration treatment is not observed.

In the concentration step, the cell concentration is preferably brought close to the limiting value of the cell concentration caused by the body length of the microalga.

According to an embodiment of the present disclosure, in the concentration step, when the microalga has a body length of less than 10 μm, concentration is preferably performed until the cell concentration reaches 2 billion cells/cc or more, more preferably performed until the cell concentration reaches 3 billion cells/cc or more, even more preferably performed until the cell concentration reaches 5 billion cells/cc or more, still more preferably performed until the cell concentration reaches 8 billion cells/cc or more, and even still more preferably performed until the cell concentration reaches 10 billion cells/cc or more.

Further, in the concentration step, when the microalgal dispersion contains nannochloropsis or chlorella, concentration is preferably performed until the cell concentration reaches 11 billion cells/cc or more, more preferably performed until the cell concentration reaches 12 billion cells/cc or more, even more preferably performed until the cell concentration reaches 14 billion cells/cc or more, and still more preferably performed until the cell concentration reaches 15 billion cells/cc.

According to an embodiment of the present disclosure, the washing method is not particularly limited. For example, the following washing method can be performed.

The microalgal dispersion is taken out from the stock solution tank 1, and the same solvent (such as seawater) as the appropriate dispersion solvent is poured over the stock solution tank 1. Thereafter, the poured liquid is separately collected, and an appropriate amount of an aqueous solution of from 30 to 500 ppm sodium hypochlorite is charged in the empty stock solution tank 1, and subjected to circulation washing, by means of the liquid feed pump 2, using a circulating line of the stock solution tank 1, the liquid feed pump 2, the hollow fiber membrane module 3, and the stock solution tank 1.

In this case, when the material of the hollow fiber membrane module 3 is cellulose acetate, circulation washing can be performed for from 1 hour to 5 hours with an aqueous solution of sodium hypochlorite having a low concentration of from 30 to 100 ppm for suppressing deterioration of the cellulose acetate membrane due to sodium hypochlorite.

As another washing method, it is also possible to periodically perform an operation generally called backwashing in which fresh water in the permeated water tank 5 is supplied to the permeated water side of the hollow fiber membrane module 3 and a backwashing liquid is fed to the stock solution tank 1.

Furthermore, the method for preparing a microalgal dispersion according to an embodiment of the present disclosure can also be carried out as a method for concentrating bacteria including microalgae (such as eubacteria or archaebacteria that do not perform photosynthesis). For example, it can be used as a method of concentrating bacteria from water to be treated to prepare a desired concentrated bacterial liquid.

### Method for Producing Valuable Product

The method for producing a valuable product according to an embodiment of the present disclosure is a method including adding and charging the microalgal dispersion (dispersion containing a microalga having a size of less than 1000 μm) produced by the method described above or a frozen product of the dispersion as a raw material, and kneading and mixing the raw material.

That is, according to an embodiment of the present disclosure, there is provided a method for preparing a microalgal dispersion for producing a valuable product containing a microalgal dispersion or a useful component produced by the microalga contained in the dispersion, or a frozen product of the microalgal dispersion or a useful component produced by the microalga contained in the frozen product.

The valuable product is selected from biofuels, pharmaceuticals, supplements, cosmetics, chemical products, foods, feeds, fertilizers, and the like. At a desired stage of a production process of each of these products, the microalgal dispersion produced by the above-described method or a frozen product thereof, or a useful component produced by the microalga contained therein can be added and charged as a production raw material, and kneaded and mixed. In this case, additives such as a dispersion stabilizer and various components required depending on the application can be added at a desired stage if necessary.

The adding and charging method may involve the use of means of solid or paste falling type, dropping type, or liquid feeding type at a predetermined addition rate via a device such as a screw, a belt conveyor, a pump, or a control valve.

The kneading and mixing method may involve the use of a stirring mixer having a stirring blade of a single shaft or a multiple shaft such as a low-speed shaft or a high-speed shaft obtained by combining a butterfly blade, an anchor blade, a paddle blade, a turbine blade, and the like, or a kneading machine such as a batch-type or continuous-type kneader or an extruder. In addition to the kneading and mixing process, sterilization treatment can be performed.

When the microalgal dispersion contains seawater and the salt content of the contained seawater is excessive, a liquid sufficiently diluted with salt-free water at any stage may be reconcentrated while being desalted by the hollow fiber membrane module.

Furthermore, when the valuable product is a biofuel, for example, the method for preparing a microalgal dispersion according to an embodiment of the present disclosure can be applied as a means in the concentration step described in JP 2011-246605 A.

Each aspect disclosed in the present specification can be combined with any other feature disclosed herein. The configurations, combinations thereof, and the like in each embodiment of the present disclosure are examples, and various additions, omissions, substitutions, and other changes of configurations may be made, as appropriate, without departing from the spirit of the disclosure of the present invention. The present disclosure is not limited by the embodiments and is limited only by the claims.

### Another Embodiment of Concentration Method of Performing Cross Flow Filtration of Microalga-Containing Liquid

Another embodiment of the concentration method of performing the cross flow filtration of the microalga-containing liquid will be described.

JP 2020-146645 A describes a method for separating and purifying a liquid containing a valuable product, wherein, when the viscosity of the liquid at 20° C. and a shear rate of 25 s⁻¹ exceeds 10 mPa's, the internal diameter of the hollow fiber membrane is preferably increased, and, when the viscosity of the liquid at 20° C. and a shear rate of 25 s⁻¹ is 30 mPa's or less, the internal diameter of the hollow fiber membrane is set to from 0.8 to 1.4 mm, and filtration is performed under conditions of a filtration pressure of from 0.08 to 0.2 MPa and a linear velocity of from 0.7 to 1.4 m/s.

Also in the method for preparing a microalgal dispersion according to an embodiment of the present disclosure, when the viscosity at 20° C. and a shear rate of 25 s⁻¹ exceeds 10 mPa·s, the internal diameter of the hollow fiber membrane is preferably increased.

When it is desired to further increase the cell concentration of the microalga, a hollow fiber membrane having a large internal diameter is advantageous for securing a high circulation linear velocity. However, when the internal diameter is increased, the membrane area per module volume decreases. Therefore, when the viscosity of the microalga-containing liquid is high, the internal diameter of the hollow fiber membrane is preferably in a range of from 1.0 to 2.0 mm, more preferably in a range of from 1.1 to 1.9 mm, and most preferably in a range of from 1.2 to 1.8 mm.

When the microalga-containing liquid has a high viscosity, the hollow fiber membrane material is less likely to be limited in terms of angle of contact with water, and a hollow fiber membrane made of polyether sulfone having an angle of contact with water of from 65° to 70° can be used.

Still another embodiment of the concentration method of performing the cross flow filtration of the microalga-containing liquid will be described. WO 22/250036 describes that alternating tangential flow (alternating cross flow) filtration can be performed in a method for separation and purification of a liquid containing a fine useful substance. Also in the method for preparing a microalgal dispersion according to an embodiment of the present disclosure, alternating tangential flow (alternating cross flow) filtration can be performed.

### EXAMPLES

### Example 1

### Method for Measuring Nannochloropsis Cell Concentration

The measuring apparatus used was a Thoma hemocytometer (HIRSCHMANN LAB. Counting Chamber 0.0025 mm²·Depth 0.100 mm).

In the measurement method, 1 liter of purified water was added to a 1-liter beaker, 1 ml of pure water was discarded with a measuring pipette, 1 ml of a nannochloropsis dispersion as a measurement sample was then added, and the nannochloropsis dispersion was diluted 1000 times.

The diluted liquid was injected into the hemocytometer, and the number of cells was counted using a hand-held counter under a microscope (200× ocular lens, 10×/objective lens, 20×).

The number of cells was measured at least three times, an average value of the numbers of cells was calculated, and the cell concentration of the nannochloropsis dispersion was determined by multiplying the average value by the dilution rate, 1000 times.

By using the apparatus illustrated in FIG. 1, 23 m³ of an aqueous solution containing nannochloropsis (solvent: seawater, cell concentration: 14 million cells/cc) was subjected to a concentration step by cross flow filtration for 21 hours using a cellulose acetate hollow fiber membrane module 3 (internal diameter: 0.8 mm, molecular weight cutoff: 150000, membrane area: 16 m²) (FN20 VP-FUC1582) under conditions of a filtration pressure of 0.06 MPa, a circulation linear velocity of 0.5 m/sec, and a liquid temperature of 26° C.

In the concentration step, the concentrated liquid was returned to a stock solution tank 1, and the permeated liquid was fed to a permeated liquid tank 5. During the treatment in the concentration step, automatic underflow washing was performed by closing an automatic on-off valve 4 for 30 seconds once every 20 minutes. No backwashing operation was performed.

The initial filtration rate during the treatment in the concentration step was 94 L/m²·Hr, and the filtration rate gradually decreased during the treatment in the concentration step, and the filtration rate after the elapse of 21 hours was 50 L/m²·Hr. The filtration rate decreased less, and the average filtration rate exceeded 60 L/m²·Hr.

The volume of the aqueous nannochloropsis solution after the treatment in the concentration step was 23 L, and the nannochloropsis cell concentration was 14.6 billion cells/cc. The concentration factor by volume was 1000 times, and the concentration factor by cell concentration was 1028 times.

After the concentrated aqueous solution containing nannochloropsis was taken out from the stock solution tank 1, 0.4 m³ of an aqueous solution of 50 ppm sodium hypochlorite was charged into the stock solution tank 1 and subjected to circulation washing from the stock solution tank through the hollow fiber membrane module to the stock solution tank 1 for 4 hours. The washing recovery rate of the hollow fiber membrane module 3 after washing did not change from the initial state at the start of the concentration treatment.

### Comparative Example 1

The concentration treatment was performed under the same conditions as in Example 1 except that the cell concentration of the aqueous solution containing nannochloropsis was 18.5 million cells/cc, the charge volume was 17 m³, the hollow fiber membrane module 3 used was a polyether sulfone hollow fiber membrane module (internal diameter: 0.8 mm, molecular weight cutoff: 150000, membrane area: 16 m²) (FN20-VP-FUS1582), and the liquid temperature was 15° C.

The initial filtration rate during the treatment in the concentration step was 94 L/m²·Hr, and the filtration rate 4 hours after the start of the treatment in the concentration step was 60 L/m²·Hr, and the filtration rate 6 hours after the start of the treatment in the concentration step was 50 L/m²·Hr. As described above, the filtration rate greatly decreased at the initial stage, and the filtration rate after the elapse of 21 hours was 31 L/m²·Hr, and the average filtration rate was significantly lower than that in Example 1.

The volume of the aqueous nannochloropsis solution after the treatment in the concentration step was 34 L, and the nannochloropsis cell concentration was 10 billion cells/cc. The concentration factor by volume was 500 times, and the concentration factor by cell concentration was 540 times.

Since the washing recovery rate of the hollow fiber membrane module was too low under the same conditions as in Example 1, the module did not return to the initial state at the start of the concentration treatment unless circulation washing with an aqueous solution of 300 ppm sodium hypochlorite for 2 hours and immersion washing with 0.1% aqueous sodium hydroxide solution for 24 hours were additionally performed.

### Referential Example 1

For clarifying the influence of the material of the hollow fiber membrane on the antifouling effect, concentration treatment (circulation linear velocity: 1.0 m/sec, operating pressure: from 0.05 to 0.06 MPa) of the same aqueous nannochloropsis solution (solvent: seawater, cell concentration: 20 million cells/cc, volume: 15 L) was performed using the same test apparatuses as small modules (cellulose acetate; FB03-VC-FUC1582) and (polyether sulfone; FB03-VC-FUS1582) having the same membrane area (0.025 m²), internal diameter (0.8 mm), and molecular weight cutoff (150000). Neither underflow washing nor backwashing operation was performed.

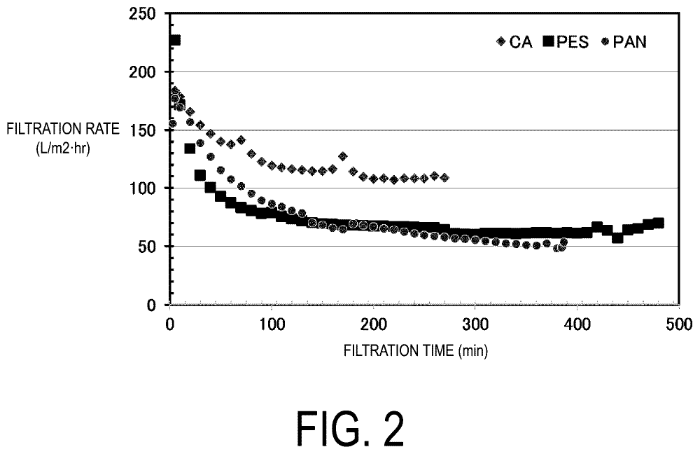

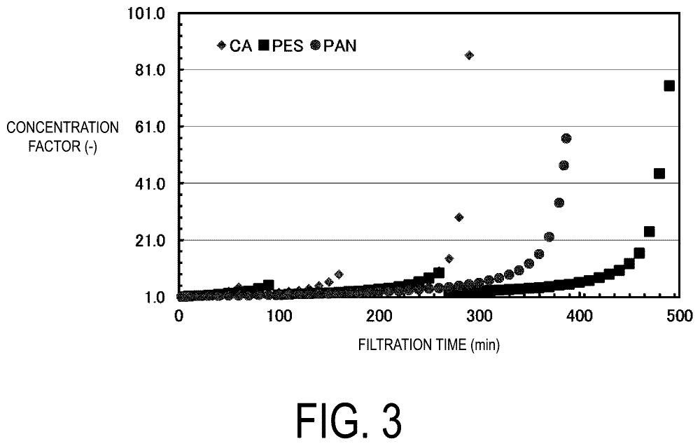

The temperature of the liquid to be treated was about 25° C. at the beginning because of no adjustment of the temperature, but increased to 35° C. at the end of the concentration treatment. The filtration rates in a period from the start to the end of the concentration treatment in the respective membrane modules were plotted together in FIG. 2, and the transitions in concentration factor were plotted in FIG. 3.

In the hollow fiber membrane module of the cellulose acetate membrane (CA), the filtration rate 5 minutes after the start of the treatment in the concentration step was 176 L/m²·Hr, and the filtration rate 270 minutes after the start of the treatment in the concentration step was 102 L/m²·Hr. The filtration rates at 280 minutes and 290 minutes after the start of the concentration treatment failed to be measured because the amount of the circulating liquid was small and bubble trapping occurred. The concentration treatment was completed 290 minutes after the start, and the liquid amount was 0.17 L. The concentration factor by volume was 88 times.

Meanwhile, in the hollow fiber membrane module of the polyether sulfone membrane (PES), the filtration rate 5 minutes after the start of the concentration treatment was 227 L/m²·Hr, but the filtration rate 40 minutes after the start of the concentration treatment was lower than 100 L/m²·Hr, and then gradually decreased. The amount of the liquid was 0.2 L after the elapse of 480 minutes.

### Referential Example 2

A concentration operation was performed using a small module (FB03-VC-FUY15E1) of an acrylonitrile hollow fiber membrane (PAN) having a membrane area (0.03 m²), an internal diameter (1.4 mm), and a molecular weight cutoff (150000) under the same conditions using the same apparatus as in Referential Example 1. The transitions in filtration rate and concentration factor in a period from the start to the end of the concentration treatment were plotted in FIGS. 2 and 3.

The initial filtration rate per unit membrane area was higher than that of the polyether sulfone membrane, and the time required for the concentration factor to rise was shorter than that of the polyether sulfone membrane by correcting the large membrane area. The aqueous nannochloropsis solution used was different in lot from that of Referential Example 1.

### Example 2 and Example 3

In the same manner as in Example 1, the concentration step by cross flow filtration of the aqueous solution containing nannochloropsis was performed by using the apparatus illustrated in FIG. 1. In Example 2, concentration was advanced in substantially the same manner as in Example 1. In Example 3, the concentration step was further advanced than in Example 2.

The solid content concentrations (mass %) and the cell concentrations (number of cells per cc) of the stock aqueous solutions containing nannochloropsis and the liquids after the concentration step treatment used in Example 2 and Example 3 were measured. The results are shown together in Table 1.

TABLE 1

| | Solid content | Number | |
|---|---|---|---|
| | concentration | of cells | |
| | (mass %) | (per cc) | |
| Stock solution (Example 2) | 0.011 | 21 | million |
| Stock solution (Example 3) | 0.015 | 22 | million |
| Concentrated liquid (Example 2) | 5.2 | 13.8 | billion |
| Concentrated liquid (Example 3) | 8 | 22.95 | billion |

### INDUSTRIAL APPLICABILITY

The microalgal dispersion according to an embodiment of the present disclosure can be used as a production raw material for biofuels, pharmaceuticals, supplements, cosmetics, chemical products, foods, feeds, and fertilizers.

### REFERENCE SIGNS LIST

1: Stock solution tank
3: Hollow fiber membrane module
5: Permeated water tank

1. A microalgal dispersion comprising a microalga having a body length of less than 10 μm, the microalgal dispersion having a cell concentration of 2 billion cells/cc or more.

2. A microalgal dispersion comprising a microalga having a body length of from 2 to 5 μm, the microalgal dispersion having a cell concentration of 10 billion cells/cc or more.

3. The microalgal dispersion according to claim 1, wherein the microalga is selected from chlorella, dunaliella, nannochloropsis, Pseudochoricystis ellipsoidea, and Cyanidioschyzon merolae.

4. A frozen product of the microalgal dispersion described in claim 1.

5. A valuable product comprising a microalgal dispersion containing a microalga having a body length of less than 1000 μm, the microalgal dispersion having a cell concentration of 2 billion cells/cc or more; a useful component produced by the microalga contained in the dispersion; a frozen product of the microalgal dispersion; or a useful component produced by the microalga contained in the frozen product, wherein the valuable product is selected from a biofuel, a pharmaceutical, a supplement, a cosmetic, a chemical product, a food, a feed, and a fertilizer.

6. The valuable product according to claim 5, wherein the microalga is a microalga having a body length of less than 10 μm.

7. A method for preparing a microalgal dispersion from a microalga-containing liquid, the microalga-containing liquid containing a microalga having a body length of less than 1000 μm, the method comprising a concentration step of performing cross flow filtration of the microalga-containing liquid using a hollow fiber membrane, wherein
a circulation linear velocity in the cross flow filtration in the concentration step is in a range of 0.2 m/sec or more and 1.3 m/sec or less.

8. The method for preparing a microalgal dispersion according to claim 7, wherein concentration is performed until a cell concentration of the microalga reaches 2 billion cells/cc or more.

9. The method for preparing a microalgal dispersion according to claim 7, wherein the microalga contained in the microalga-containing liquid is a microalga having a body length of less than 500 μm and selected from spirulina, chlorella, euglena, pavlova, haematococcus, dunaliella, nannochloropsis, phaeodactylum, porphyridium, labyrinthula, algae belonging to the genus Pseudochoricystis (Pseudochoricystis) including Pseudochoricystis ellipsoidea (Pseudochoricystis ellipsoidea); algae belonging to the genus Choricystis (Choricystis); algae belonging to the genus Chlamydomonas (Chlamydomonas) including Chlamydomonas reinhardtii (Chlamydomonas reinhardtii); algae belonging to the genus Cyanidioschyzon (Cyanidioschyzon) including Cyanidioschyzon merolae (Cyanidioschyzon merolae), or algae belonging to the genus Cyanidium (Cyanidium) including Cyanidium caldarium (Cyanidium caldarium).

10. The method for preparing a microalgal dispersion according to claim 7, wherein the microalga contained in the microalga-containing liquid is a microalga having a body length of less than 10 μm and selected from chlorella, dunaliella, nannochloropsis, Pseudochoricystis ellipsoidea, and Cyanidioschyzon merolae.

11. The method for preparing a microalgal dispersion according to claim 7, wherein the hollow fiber membrane is a hollow fiber membrane made of an inorganic material or an organic polymer material having an angle of contact with water of less than 60°.

12. The method for preparing a microalgal dispersion according to claim 11, wherein the hollow fiber membrane made of an organic polymer material having an angle of contact with water of less than 60° is an ultrafiltration membrane having an internal diameter in a range of from 0.5 mm to 2.0 mm and a molecular weight cutoff in a range of from 10000 to 300000.

13. The method for preparing a microalgal dispersion according to claim 7, wherein an operating pressure at an inlet in the cross flow filtration in the concentration step is in a range of from 0.05 MPa to 0.2 MPa, and an average filtration rate is in a range of from 50 L/m²·Hr to 150 L/m²·Hr.

14. A method for producing a valuable product, the method comprising producing a microalgal dispersion by the method for preparing a microalgal dispersion described in claim 7; adding and charging the microalgal dispersion as a production raw material; and kneading and mixing the production raw material, wherein
the valuable product is selected from a biofuel, a pharmaceutical, a supplement, a cosmetic, a chemical product, a food, a feed, and a fertilizer.

15. The method for producing a valuable product according to claim 14, wherein the production raw material is a frozen product of the microalgal dispersion.

16. A valuable product comprising:
a microalgal dispersion containing a microalga having a body length of less than 1000 μm, the microalgal dispersion having a solid content concentration of 5% or more;
a useful component produced by the microalga contained in the dispersion;
a frozen product of the microalgal dispersion; or
a useful component produced by the microalga contained in the frozen product,
wherein the valuable product is selected from a biofuel, a pharmaceutical, a supplement, a cosmetic, a chemical product, a food, a feed, and a fertilizer.

17. A method for producing a valuable product, the method comprising:
adding and charging, as a production raw material,
a microalgal dispersion containing a microalga having a body length of less than 1000 μm, the microalgal dispersion having a solid content concentration of 5% or more,
a useful component produced by the microalga contained in the dispersion,
a frozen product of the microalgal dispersion, or
a useful component produced by the microalga contained in the frozen product; and
kneading and mixing the production raw material.
